(19) United States
(12) Patent Application Publication
(10) Pub. No.: US 2025/0272794 A1
(43) Pub. Date: Aug. 28, 2025

(54) SYSTEMS AND METHODS FOR MRI CONTRAST SYNTHESIS UNDER LIGHT-WEIGHTED FRAMEWORK

(71) Applicant: Subtle Medical, Inc., Menlo Park, CA (US)

(72) Inventor: Long WANG, Menlo Park, CA (US)

(21) Appl. No.: 19/209,044

(22) Filed: May 15, 2025

Related U.S. Application Data
(63) Continuation of application No. PCT/US2023/080243, filed on Nov. 17, 2023.
(60) Provisional application No. 63/384,888, filed on Nov. 23, 2022.

(51) Int. Cl. G06T 5/50; G06T 5/60; G06T 5/77; G06T 5/94; G06T 7/215; G06V 10/25; G06V 10/764; G06V 10/774; G06V 10/82

(52) U.S. Cl. CPC G06T 5/50; G06T 5/60; G06T 5/77; G06T 5/94; G06T 7/215; G06V 10/25; G06V 10/764; G06V 10/774; G06V 10/82; G06T 2207/10088; G06T 2207/20081; G06T 2207/20084; G06T 2207/20221; G06V 2201/03

(57) ABSTRACT

Methods and systems are provided for synthesizing a contrast-weighted image in Magnetic resonance imaging (MRI). The method comprises: receiving a multi-contrast image of a subject, where the multi-contrast image comprises one or more images of one or more different contrasts; and generating, by a deep learning model, a synthesized image having a target contrast that is different from the one or more different contrasts of the one or more images. The deep learning model is trained by a framework comprising a segmentation network for generating a segmentation map, a classification network for generating a pathology aware map and a reconstruction network for generating a plurality of synthesized images with different brightness levels in a tissue area.

### CROSS-REFERENCE TO RELATED APPLICATION

This application is a continuation of International Application No. PCT/US2023/080243 filed Nov. 17, 2023, which claims priority to U.S. Provisional Application No. 63/384,888 filed on Nov. 23, 2022, the content of which is incorporated herein in its entirety.

### BACKGROUND

Magnetic resonance imaging (MRI) has been used to visualize different soft tissue characteristics by varying the sequence parameters such as the echo time and repetition time. Through such variations, the same anatomical region can be visualized under different contrast conditions and the collection of such images of a single subject is known as multi-contrast MRI. Multi-contrast MRI provides complimentary information about the underlying structure as each contrast highlights different anatomy or pathology. For instance, complementary information from multiple contrast-weighted images such as T1-weighted (T1), T2-weighted (T2), proton density (PD), diffusion weighted (DWI) or Fluid Attenuation by Inversion Recovery (FLAIR) in magnetic resonance imaging (MRI) has been used in clinical practice for disease diagnosis, treatment planning as well as down-steam image analysis tasks such as tumor segmentation. Each contrast provides complementary information. As an example, T1 weighted (T1w), T2 weighted (T2w), and short tau inversion recovery (STIR) multi-contrast imaging are commonly used in routine clinical practice to detect pathological processes for MR spine scans. However, the prolonged total scan time for a full MR exam (full MR exam for spine performing the multi-contrast imaging can take 20-40 minutes) can result in image artifacts or misalignment issues for clinical diagnosis.

### SUMMARY

The present disclosure addresses the above needs by providing a reconstruction network integrated the anatomy and pathology information in the synthesis process. In particular, the entire pipeline may be lightweight and yet combined all the essential information with the anatomies or pathologies. The entire scanning time may be significantly reduced or the contrast agent dose level may be reduced by synthesizing a contrast-weighted image in Magnetic resonance imaging (MRI) based on other acquired contrast-weighted images. For example, in order to reduce scanning time, only selected contrasts are acquired while other contrasts are ignored. In another example, one or more of the multiple contrast images may have poor image quality that are not usable or lower quality due to reduced dose of contrast agent. The provided methods and systems may synthesize a missing contrast-weighted image based on other contrast images. Deep learning (DL) image synthesis may be employed for synthesizing a missing or low-quality contrast-weighted image based on other available images with different contrasts, allowing for faster MR acquisitions while matching or exceeding routine standard of care (SOC) quality.

In an aspect, methods and systems are provided for synthesizing a contrast-weighted image in Magnetic resonance imaging (MRI). The method comprises: receiving a multi-contrast image of a subject, where the multi-contrast image comprises one or more images of one or more different contrasts; and generating, by a deep learning model, a synthesized image having a target contrast that is different from the one or more different contrasts of the one or more images. The deep learning model is trained by a framework comprising a segmentation network for generating a segmentation map, a classification network for generating a pathology aware map and a reconstruction network for generating a plurality of synthesized images with different brightness levels in a tissue area.

In another aspect of the present disclosure a computer-implemented method for synthesizing a contrast-weighted image is provided. The method comprises: (a) receiving a multi-contrast image of a subject, wherein the multi-contrast image comprises one or more acquired images of one or more different contrasts, and wherein the one or more different contrasts correspond to one or more different pulse sequences for acquiring the multi-contrast image; (b) generating an input data to be processed by a deep learning model, where the deep learning model is trained using a training data pair, where the training data pair includes an input image or a ground truth image, and wherein the input image or the ground truth image is adjusted based on a segmentation of a region of interest (ROI); and (c) generating, by the deep learning model, a synthesized image based on the input data, where the synthesized image has a target contrast that is different from the one or more different contrasts of the one or more acquired images.

In a related yet separate aspect, non-transitory computer-readable storage medium including instructions that, when executed by one or more processors, cause the one or more processors to perform operations for synthesizing a contrast-weighted image is provided. The operations comprise: (a) receiving a multi-contrast image of a subject, wherein the multi-contrast image comprises one or more acquired images of one or more different contrasts, and wherein the one or more different contrasts correspond to one or more different pulse sequences for acquiring the multi-contrast image; (b) generating an input data to be processed by a deep learning model, where the deep learning model is trained using a training data pair, where the training data pair includes an input image or a ground truth image, and wherein the input image or the ground truth image is adjusted based on a segmentation of a region of interest (ROI); and (c) generating, by the deep learning model, a synthesized image based on the input data, where the synthesized image has a target contrast that is different from the one or more different contrasts of the one or more acquired images.

In some embodiments, generating the input data comprises registering the one or more acquired images. In some cases, registering the one or more acquired images comprises adjusting at least one of the one or more acquired images based on a segmentation of a ROI. In some instances, adjusting the at least one of the one or more acquired images comprises replacing the ROI in the at least one of the one or more acquired images with the segmentation of the ROI. In some examples, the ROI contains motion of an anatomical region within the ROI.

In some embodiments, the deep learning model is trained by a framework comprising a segmentation network for generating a segmentation map, and a classification network for generating a pathology map. In some cases, the segmentation map, and the pathology map are used to train the deep learning model. In some cases, the ground truth image is adjusted by generating different brightness levels in a tissue area based on the segmentation of the ROI generated by the segmentation network. In some cases, the input image of the training data pair is adjusted by replacing the ROI in the input image based on the segmentation of the ROI. In some cases, the segmentation map is embedded into a loss function to train the deep learning model. In some cases, the pathology map is embedded into a loss function to train the deep learning model. In some embodiments, the multi-contrast image is acquired using a magnetic resonance (MR) device.

Additional aspects and advantages of the present disclosure will become readily apparent to those skilled in this art from the following detailed description, where only illustrative embodiments of the present disclosure are shown and described. As will be realized, the present disclosure is capable of other and different embodiments, and its several details are capable of modifications in various obvious respects, all without departing from the disclosure. Accordingly, the drawings and descriptions are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example of the architecture of the network.

FIG. 2 shows examples of different brightness levels for the fat.

FIG. 3 shows a comparison of an acquired STIR image (left) and a synthesized STIR image (right).

FIG. 4 shows a comparison of an acquired T1 image (left) and a synthesized T1 image (right).

FIG. 5 shows a comparison of an acquired T2 image (left) and a synthesized T2 image (right).

FIG. 6 and FIG. 7 shows Bland-Altman plots of the experiment results.

FIG. 8 and FIG. 9 show the distribution plot of differences between the acquired STIR and Synthesized STIR.

FIG. 10 and FIG. 11 show examples of Passing-bablok regression plot for each tissue.

FIG. 12 schematically illustrates a system implemented on an imaging platform for performing one or more methods/algorithms described herein.

FIG. 13 shows an example of abdomen swap in the input data.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Multi-contrast MRI provides complimentary information about the underlying structure as each contrast highlights different anatomy or pathology. By varying the sequence parameters such as the echo time and repetition time, the same anatomical region can be visualized under different contrast conditions and the collection of such images of a single subject is known as multi-contrast MRI. For example, MRI can provide multiple contrast-weighted images using different pulse sequences and protocols (e.g., T1-weighted (T1), T2-weighted (T2), proton density (PD), diffusion weighted (DWI), Fluid Attenuation by Inversion Recovery (FLAIR), tau inversion recovery (STIR), and the like in magnetic resonance imaging (MRI)). These different multiple contrast-weighted MR images may also be referred to as multi-contrast MR images. In some cases, one or more contrast-weighted images or MRI sequences may be missing or not available. For example, in order to reduce scanning time, only selected contrasts are acquired while other contrasts are ignored. In another example, one or more of the multiple contrast images may have poor image quality that are not usable or lower quality due to reduced dose of contrast agent. It may be desirable to synthesize a missing contrast-weighted image based on other contrast images.

Methods and systems herein may provide a deep learning-based algorithm for synthesizing a contrast image in Magnetic resonance imaging (MRI). In particular, the synthesized MR image may be generated based on input MR images acquired under different contrast conditions/sequences thereby reducing the MR scanning time. For example, the output may be a synthesized short tau inversion recovery (STIR) image and the input images may be contrast-weighted images using different pulse sequences and protocols (e.g., T1-weighted (T1), T2-weighted (T2)). The output and input can be any contrast sequences. For example, the input may be STIR and T1-weighted image and the output may be synthesized T2-weighted image. In another example, the input may be STIR and T2-weighted image and the output may be synthesized T1-weighted image.

Systems and methods herein may be used to synthesize a “missing contrast” which is a contrast need to be synthesized due to various reasons such as low quality (not usable), or not available (e.g., not acquired to shorten scanning time). Depending on specific tissues or organs are imaged, different contrast images may be acquired or synthesized. The term “contrast” as utilized herein may refer to an MRI sequence such as T1-weighted (T1), T2-weighted (T2), proton density (PD), diffusion weighted (DWI) or Fluid Attenuation by Inversion Recovery (FLAIR) and other MRI sequences. For example, images of different contrasts may refer to images acquired with different pulse sequences.

### Network Architecture

Systems herein may be used for generating a full exam. A full exam or full scanning procedure may include a plurality of contrasts, sequences, or a plurality of series such as Sagittal (sag) T1, Sag T2, Sag STIR, Axial T1, and Axial T2. However, performing full exam can be lengthy and costly. The system herein may beneficially shorten the duration or timing of the exam procedure by acquiring a subset of the contrasts/series from the full exam contrasts/series while synthesizing the remaining (missing or targeting) subset of contrasts. Methods and systems herein can also be used to replace a contrast sequence that has a low quality or improve the quality of a contrast sequence that has a low quality.

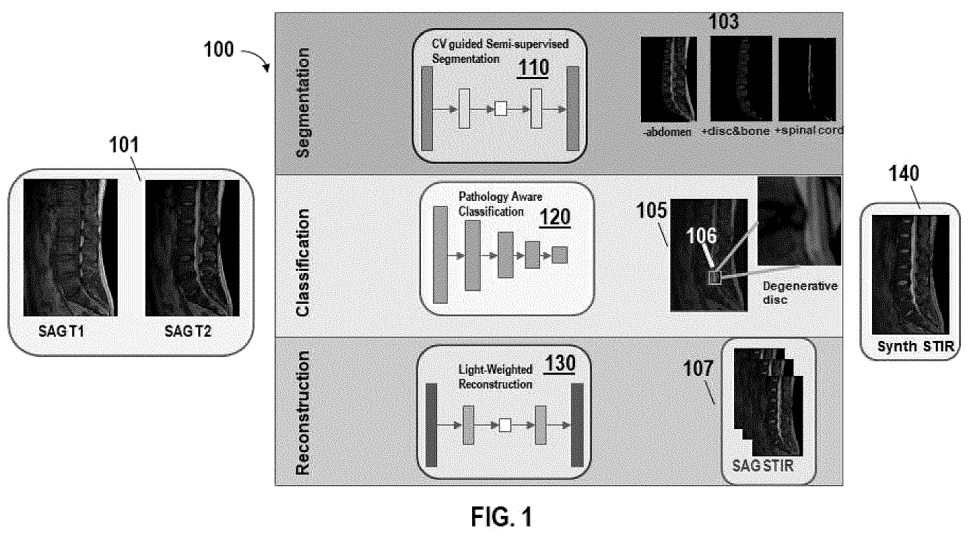

FIG. 1 shows an example of the architecture of the network 100. In some embodiments, the input image 101 may comprise one or more different MRI contrast images (e.g., T1, T2, etc.). The one or more different MRI contrast images may include images of different contrasts or sequences (e.g., T1, T2, etc.). The different contrasts or sequences may be acquired in the same imaging session. For example, the input images may include sagittal (sag) T1 and Sag T2 images of the same patient and same target region/anatomies. In some cases, the input images may have contrasts different from that of the synthesized output image 140. The input images may have any combination of different contrasts or sequences. For example, the input images may have a combination of T1 and T2, a combination of T1 and STIR, whereas the synthesized/predicted output image may be STIR and T2 respectively.

The framework 100 may comprise a plurality of components such as a segmentation network (e.g., anatomy segmentation network) 110, a classification network (e.g., pathology aware classification) 120, and a reconstruction network (e.g., light-weighted reconstruction network) 130. In some embodiments, the architecture 100 may comprise a reconstruction network 130 integrated the anatomy information from the segmentation network 110 and pathology information from the classification network 120 in the synthesis process.

In some cases, the output of the model network 100 may comprise multiple channels. For example, a five-channel output may be predicted by the model. In some embodiments, the multiple channels may comprise at least a segmentation map 103, a weight map 105 for the pathology and a synthesized/predicted image of a contrast different than the input images. As illustrated in FIG. 1, the output may comprise a plurality of synthetic STIR (e.g., three synthetic STIR images with different levels of brightness in fat) 107, a segmentation map for each anatomy 103, and a weight map 105 for the pathology. It should be noted that other number of channels can be included. For example, when two, three, four, five, six or more synthetic STIR images are generated, the number of output channels may be four, five, six, seven, eight or more.

The segmentation map and the weight map may be applied in a training process of the reconstruction network and served an important factor in the loss function. Details about the training process and training data are described later herein. After the reconstruction model 130 is trained using the segmentation map 103, weight map 105, and one or more STIR images of one or more brightness levels as ground truth (along with the input T1, and T2), the model may be deployed to make inference. For example, the model 130 may be trained to make an inference or generate a synthetic STIR image 140 based on input T1 and T2 images 101.

### Anatomy Aware Map

As described above, the framework 100 of the present disclosure may comprise an anatomy segmentation network 110. The systems and methods herein may provide an anatomy segmentation network 110 to extract one or more masks for one or more anatomies/anatomical regions or region of interest (ROI) (e.g., abdomen, fat, and spinal cord, etc.) and generate an anatomy aware map or segmentation map based on the one or more masks. The number of masks or the one or more anatomies to be extracted may be determined based on the body part under the imaging/examination. In some embodiments, the anatomy segmentation network 110 may utilize computer vision based semi-supervised method to extract the one or more masks for the one or more anatomical regions such as abdomen, fat, and spinal cord from the input image 101, and may summarize them into one segmentation map 103.

In some cases, during a training process of the framework 100 or the reconstruction network, the methods herein may include one or more unique adjustments applied to the training process, the training dataset or a preprocessing of the input data by utilizing the one or more masks or segmentations (e.g., abdomen image, the spinal cord image, and the fat image) generated by the segmentation network.

In some embodiments, at least one of the one or more segmentations or masks may be utilized to preprocess or improve a quality of the training data (during the training phase) or the input data (during the interference phase). For instance, during the training process, the abdomen region of the acquired input images may be adjusted. In some cases, the input image for training the network model or during the inference may comprise multiple contrast-weighted images from multiple scans. The cross-contrast images of multiple contrasts (e.g., T1, T2, STIR, etc.) may be pre-processed such as by registration using a registration algorithm to form a pair of registered multi-contrast images. For example, image/volume co-registration algorithms may be applied to a pair of T1 and T2 images, a pair of T1 and STIR images or a pair of T2 and STIR images to generate spatially matched images/volumes. In some cases, the co-registration algorithms may comprise a coarse scale rigid algorithm to achieve an initial estimation of an alignment, followed by a fine-grain rigid/non-rigid co-registration algorithm. However, certain region such as abdomen region may have motion during a scanning process (due to movement of abdomen). The method herein may replace a region of interest (ROI) containing motion of an anatomical feature within the ROI in an original input to improve the registration result. When registering one scan of a first contrast to another scan of another contrast, the motion area may be mismatched thus resulting in the model generating smooth/blurry results. In some cases, an anatomical region with motion (e.g., abdomen region) may be swapped/replaced for fixing registration thereby improving the efficiency in the registration process. The methods herein may improve the registration process and prevent the smoothness by swapping out a motion region (e.g., abdomen region) using a segmentation mask or segmentation of the motion region. The registration with the unique anatomical region adjustment may be performed prior to training to generate better training data pairs or prior to inference to improve the quality of the input images.

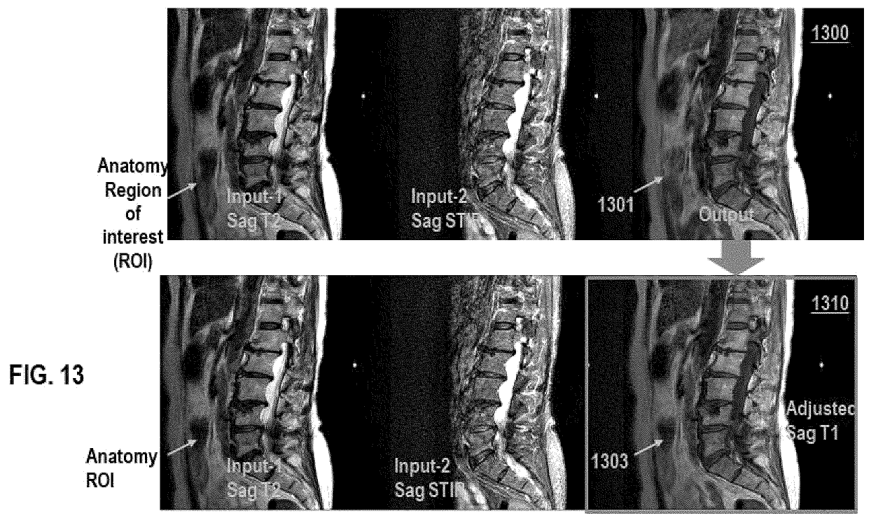

FIG. 13 shows an example of swapping a region with motion (e.g., abdomen region) in a pair of input images. As described above, the adjustment may be applied to a pair of input images of different contrasts. For example, an abdomen mask (e.g., a binary mask) is extracted with the two input images input-1 Sag T2 and input-2 Sag STIR (or other inputs such as input-1 Sag T1 and input-2 Sag T2). Next, the mask is applied on both the input images such as the Sag T2 and Sag STIR images (or Sag T1 and Sag T2 images) to obtain the segmentation area for both Sag T2 and Sag STIR (or T1 and T2). In some cases, the method may further comprise equalizing the contrast level of the two segmentation images such as by utilizing histogram equalization to make the two segmented Sag T2 and Sag STIR images (or Sag T1 and Sag T2 images) having the same contrast level. For example, when the target image to be adjusted is Sag T2, segmented abdomen from Sag STIR may be adjusted to the same contrast level as T2, and the original Sag T2's abdomen region is replaced with the adjusted abdomen segmentation from Sag STIR. The adjusted T2 (or T1) image with the replaced abdomen may beneficially help to avoid the effects from abdomen movement during a registration process.

In some cases, the adjustment of a ROI with motion may be applied to the ground truth image during the training process. For example, as shown in FIG. 13, when the input images are input-1 Sag T2, and input-2 Sag STIR, and the ground truth image includes Sag T1 (e.g., output Sag T1) 1300, the abdomen region 1301 in the output Sag T1 1300 may be replaced with the adjusted segmentation 1303 to form the adjusted Sag T1 1310. The adjusted Sag T1 may then be used as ground truth and is registered with the input images input-1 Sag T2, and input-2 Sag STIR to form a training data pair.

In some cases, during the inference stage (after the reconstruction network is developed and deployed for making inferences), the one or more unique adjustments may also be applied to preprocessing of the input data to the reconstruction network by utilizing the one or more masks or segmentations (e.g., abdomen image, the spinal cord image, and the fat image) generated by the segmentation network. Alternatively, during an inference stage, unlike the training process, the adjustment of the ROI with motion (e.g., abdomen region) is not performed in the preprocessing of the input image, as the standard of care may not be available (e.g., only one input image is available), and the reconstruction model is trained to learn the direct mapping from the input1 to the target in the ROI with motion (e.g., abdomen region).

In some embodiments, at least one of the one or more segmentations or masks may be utilized to adjust a training algorithm. For example, one or more segmentations generated by the segmentation network (e.g., a spinal cord image segmentation) may be embedded into the loss function directly for training the reconstruction network. For instance, a greater/larger weight may be applied in the spinal cord area, and an edge loss may be applied to preserve the edge structure for the spinal cord root.

In some embodiments, at least one of the one or more segmentations or masks may be utilized to adjust brightness levels of an anatomical area to allow for synthesized images with different brightness levels. Short tau inversion recovery (STIR), also known as short T1 inversion recovery, is a fat suppression technique, where the signal of fat is zero. However, due to factors such as partial volume effects or field inhomogeneities, the fat signals in the acquired STIR may not be well suppressed. In some cases, during the training process, multiple ground truth or reference STIR images having different brightness levels in the fat area may be used to train the model which beneficially allows for, later in the inference stage, customizing the fat brightness based on the need. For example, segmentation of the fat area may be used to generate customized output in the fat area. In some cases when the training pairs are prepared, the alpha matting is applied to customized brightness level in a target output (the standard of care). To reduce potential artifacts from the matting boundary, the method herein may be capable of automatically identifying regions such as the fat/air area and abdomen area for the brightness level adjustment. The output of multiple target STIR channels (e.g., three, four, five or more) may have different levels of brightness on the identified anatomies. Such multiple brightness levels beneficially allow the model to learn to reconstruct different brightness level on the outputs during the training process. In the phase of inference, the reconstruction channels are with different brightness on the identified anatomies, and only those channels are utilized for the following post-processing steps.

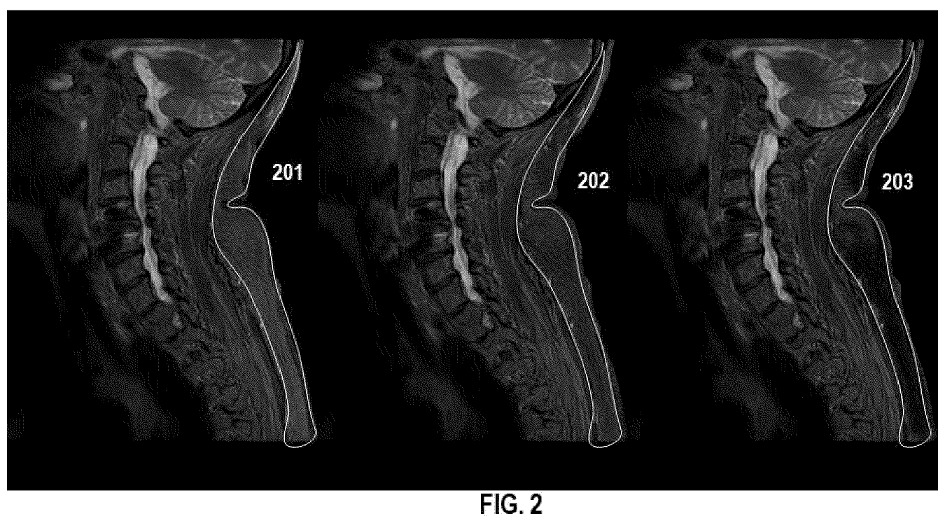

For instance, a fat mask may be applied on the multiple ground truth or reference images to generate multiple brightness levels. Such ground truth or references images with the adjusted fat areas may be included as part of the multiple channels input for training the reconstruction network. FIG. 2 shows examples of different brightness levels 201, 202, 203 for the fat area. The examples illustrated in FIG. 2 may be reference images or adjusted ground truth image including a first reference image with the highest brightness level for the fat area 201, a second reference image with the medium brightness level for the fat area 202, and a third inference image with the minimal brightness level for the fat area 203. In some cases, an inference result generated by the model may also include a first synthesized image with the highest brightness level for the fat area 201, a second synthesized image with the medium brightness level for the fat area 202, and a third synthesized image with the minimal brightness level for the fat area 203. It should be noted that though three different brightness levels are described, there can be any other number of intermediate brightness levels.

### Pathology Aware Map

As described above, the framework 100 of the present disclosure may comprise a classification model 120 for pathology aware classification. The systems and methods herein may provide a classification model 120 trained to generate a pathology aware map containing disease pathology information for training the reconstruction network. The pathology aware map beneficially improves the visibility of each pathology area.

The pathology aware map may be used for training the reconstruction network. For instance, the pathology aware map and the segmentation map may be concatenated with the original output images and passed on to the deep learning network (reconstruction network) for training the model. In some cases, the segmentation model 110 and the pathology classification model 120 may be trained before training the reconstruction model. In the training phase of the reconstruction model, the pathology aware map and the segmentation map generated by the segmentation model 110 and the pathology classification model 120 respectively may be utilized in the loss function and guide the model with information on the anatomies and pathologies. In some cases, once the reconstruction network 130 is trained and during the inference phase of the reconstruction model for generating the synthetic image 140, only the reconstruction model is utilized for the image synthesis.

In some cases, the input for generating the pathology aware map may be the same input images (e.g., T1 and T2 image) 101 and the output may be a map (e.g., heatmap) with areas indicating pathologies i.e., saliency map. For example, the heatmap (105 of FIG. 1) may highlight areas that are likely to be pathologies (e.g., the highlight region 106 displayed in FIG. 1). Depending on the body part (e.g., spine) or pathology (e.g., spine pathologies), the highlighted area may include, for example, most of the spine pathologies (including the trauma, cord lesion, non-cord lesion, degenerative disc disease, and infections). The classification model 120 may comprise any suitable machine learning or deep learning mechanism capable of identifying regions of interest (ROI) such as lesions or areas containing pathology on the images.

### Light-Weighted Reconstruction Network

The framework 100 of the present disclosure may comprise a reconstruction network 130 for generating the synthesized image 140. Depending on the body part, the synthesized image may include multiple synthesized images with multiple brightness levels (e.g., multiple STIR images with multiple brightness levels 107).

In some embodiments, in the phase of training the reconstruction network, the training data may comprise i) the input data to the reconstruction network such as the acquired/original input images with different contrasts/sequence (e.g., T1 and T2 images), and ii) the target data including the ground truth image or target image (e.g., multiple STIR images of multiple brightness levels) and the segmentation map and the saliency map. The segmentation map and saliency map may be used for model training only. Multiple brightness levels for the STIR images may be derived from the mixed effect from the target STIR images and the anatomies segmentation map. In some cases, the target data may have multiple channels including one or more channels for the ground truth image (e.g., multiple STIR images of multiple brightness levels) and two additional channels for the segmentation map and saliency map.

Once the reconstruction network is trained, in the phase of testing or inference, only the reconstruction model is utilized for the image synthesis or for generating the synthetic image. The segmentation map and saliency map may be used for model training only and may not be generated in the inference phase.

In some embodiments, to improve the inference efficiency, a light-weighted U-Net based structure 130 may be used during the training. The UNet-based network structure may be a U-shaped self-encoder structure comprising an encoder path and a decoder path. The encoder path is used to progressively extract features of the input image and progressively lower the feature level. The decoder path gradually restores the features to the resolution of the original input image by means of upsampling and jump connections and generates the synthesized image. The UNet may comprise a jump connection, i.e. a connection of a feature in the encoder path with a feature in the corresponding decoder path. The jump connection may allow the network to better utilize the characteristic information of different levels, thereby improving the accuracy and detail retaining capability of segmentation. In some cases, the UNet may comprise a downsampled block (encoder block) consisting of two convolutional layers, a ReLU activation function, and a max-pooling layer. The decoder path is composed of an upsampling layer, a convolution layer and a jump connection. At the final output layer, a convolution layer classified pixel by pixel is typically employed to output the segmentation result of the same size as the input image.

In some embodiments, UNet related variants may be utilized as the reconstruction network for taking the full input images as an input to the model and utilizing the global information of the full input image. Comparing with other network structures such as ResNet, Diffusion Network, or Transformer Network, the light-weighted U-Net may have fewer network parameters and is suitable when the computational resources are limited. Compared with the classical UNet, the variants of the light-weighted U-Net may include using large convolution kernels and depth-wise separable convolution to reduce the FLOPs (and model parameters) and introducing additional attention mechanism to improve model efficiency and reduce the effects from irrelevant features.

In some cases, one or more input images (e.g., Sag T1, Sag T2) 101 may be fed into the network and a multiple-channel output (e.g., 2D outputs) may be generated. In some cases, in the training phase, the output may comprise a stack of synthesis results (multiple channels with different brightness levels) and two maps (i.e. the anatomies segmentations map and the pathology map). The two maps are utilized in the loss function to guide the training. In the test or inference phase, the output may comprise the same number of channels as that for training, but only the channels for the synthesis images are used for ensemble and the following postprocessing. In some cases, the input images may be concatenated after passing through the encoder phase. This beneficially mitigates the issues from the misregistration (e.g., areas of moved spinal cord root or abdomen). Next, the concatenated vector may be passing through the decoder and return the output with multiple channels.

The loss function may be a mixed function for all channels except anatomies segmentation map and the heatmap for the pathologies. In some cases, the heatmap/saliency map (or segmentation map) may be weighted and added into the loss function to guide the network learning the features.

As described above, the output of the model during training may have multiple channels. For example, the output may have five channels including three channels for the generated/reconstructed STIR images (three brightness levels) which is used for image ensemble, and the two channels for the segmentation map and saliency map are used for embedding in the loss function. In some cases, the image ensemble may employ weighted averaging strategy such as assigning weights to each channel of the synthesis image and then combining all of the weighted channels to create a new image (e.g., the sum of all weights is 1). An example of the loss function is illustrated as following: for the three channels for the reconstructed images (e.g., reconstructed STIR images with three brightness levels), the image-wise reconstruction using a mixed loss of L1, SSIM, and DISTS are applied:

$${L}_{1} = {‖ {I}_{recon} - {I}_{soc} ‖}^{1}$$

$${L}_{ssim} = 1 - SSIM ( {I}_{recon} - {I}_{soc} )$$

$${L}_{DISTS} = DISTS ( {I}_{recon} , {I}_{soc} )$$

$${L}_{image} = α {L}_{1} + β {L}_{ssim} + γ {L}_{DISTS}$$

Here I_{recon} and I_{soc} are the reconstructed and ground truth images, respectively. L₁ is the L1 loss, Lₛₛᵢₘ is the Structural Similarity Index (SSIM) loss, and L_{DISTS} is the DISTS loss. The weights α, β, and γ are used to balance the contribution of each loss to the total image loss. For the two channel for the segmentation mask and saliency map, the weighted mask-based loss are applied. It can be represented as:

$${L}_{mask} = {\sum }_{i} {\sum }_{j} {w}_{i , j} {‖ {M}_{recon} ( i , j ) - {M}_{soc} ( i , j ) ‖}^{2}$$

In this equation, M_{recon} (·) and M_{soc}(·) are the reconstructed and ground truth masks, respectively. w_{i,j} is the weight associated with the pixel at location (i,j). This loss may focus on the mask area by assigning different weights to the pixel value at different positions.

### Robust Pipeline

In some embodiments, the training pipeline may comprise multiple phases. In some cases, a first phase may comprise utilizing a computer vision based semi supervised method or the segmentation network 110 to extract one or more masks for one or more ROIs (e.g., abdomen, fat, and spinal cord), and summarize the masks into a segmentation map (segmentation map). In some cases, a second phase may comprise training the pathology classification model 120 from image to the respective pathology, and extracting the pathology information into a saliency map. In optional cases such as spine pathology, a fat mask may be applied on the reference images or ground truth image to generate adjusted ground truth images with multiple brightness levels. The training data including the multiple channels of the segmentation map, pathology map/saliency map and the ground truth image (e.g., five channels including the segmentation map, pathology map, and multiple images with different brightness levels for the fat area) may be used for training the reconstruction network.

During an inference stage, unlike the training process, the adjustment of the abdomen region is not needed in the preprocessing of the input image, because there is not standard of care available, and the reconstruction model is trained to learn the direct mapping from the input1 to the target in the abdomen area. In some cases, during the inference phase, the methods herein may include additional preprocessing steps for improving the input image data quality thereby improving a prediction or inference result. For instance, to get a descent reconstruction performance, operations may be performed such that the two sequences/contrasts of the input images acquired from two scans are under the same field of view (FOV) with the same pixel spacing and/or the two sequences are registered. For example, a user may load the acquired input images in the dicom format (e.g., dicom array), affine matrix, position offsets, and voxel size, identify the FOV intersection region for cropping, and then convert a common FOV position to coordinate on the reference image (i.e. the input-1 or the input-2 designated as reference), so that the two scans are under the same FOV. In some cases, a rigid registration may also be performed such as using SimpleElastic to ensure the two scans (sequences) are matched. In some cases, the inference process may further comprise an operation to compensate the signal noise ratio (SNR) variances. For example, an additional denoising filter may be selected to be enabled so that the pipeline is robust with images under variance noise level.

### EXPERIMENT AND EXAMPLES

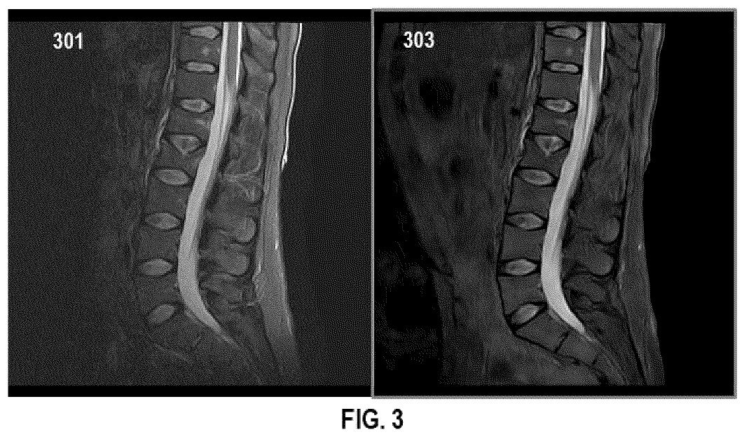

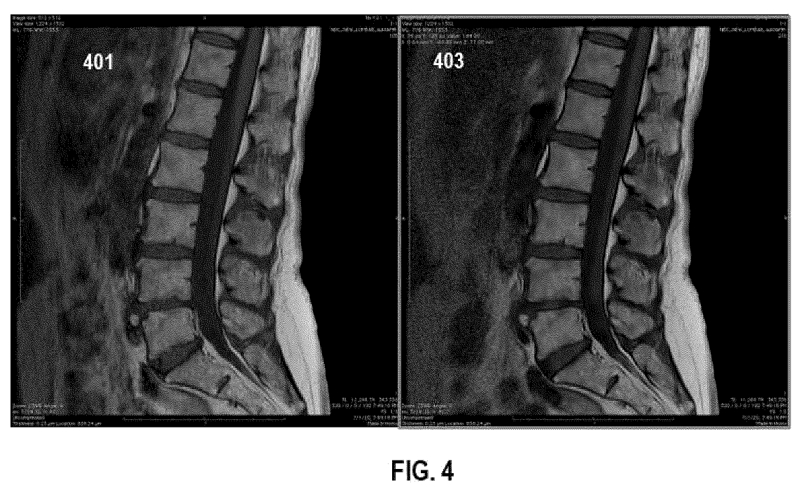

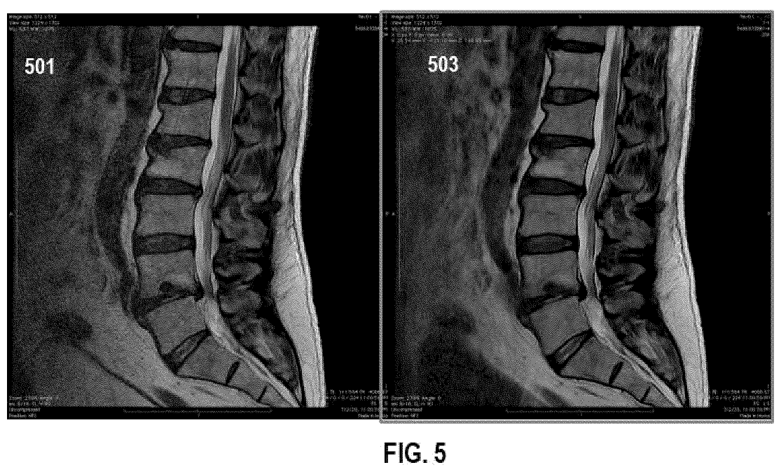

The model performance is evaluated under two aspects including interchangeability of the disease, and the pixel level consistency. Interchangeability of the disease indicates how well the model can be applied to different diseases. For the interchangeability evaluation, 80 patients with various pathologies were recruited and scanned using different scanners from multiple sites, and 30 patients were recruited for a trauma specific studies. The whole study (with either Synth STIR or acquired STIR) were reviewed by 5 radiologists with different specificities. The datasets were blinded and randomized in sequence. The result of the two-way model of ICC analysis demonstrated that the decrease in inter-reader agreement expected by randomly introducing AI-generated STIR images was 0.94% (CI [−3.53, 5.22]), which indicates that STIR images generated by the provided model (AI-generated STIR images) are significantly interchangeable with tradi4onal STIR images. Meanwhile, a Wilcoxon signed-rank test showed a significantly higher median IQ score for AI-generated STIR images compared to traditional STIR images (median difference=0; P<0.0001), and a t-test was also performed on the paired difference between IQ scores for AI-generated versus tradi4onal STIR images, and this showed a significantly higher mean IQ score for AI-generated STIR images compared to traditional STIR images (average paired difference=0.37, 95% CI [0.25, 0.49], P<0.0001). FIG. 3 shows a comparison of an acquired STIR image (left) 301 and a synthesized STIR image (right) 303. The synthesized STIR image is generated based on the input image of T1 and T2. FIG. 4 shows a comparison of an acquired T1 image (left) 401 and a synthesized T1 image (right) 403. The synthesized T1 image is generated based on the input image of T2 and STIR. FIG. 5 shows a comparison of an acquired T2 image (left) 501 and a synthesized T2 image (right) 503. The synthesized T2 image is generated based on the input image of T1 and STIR.

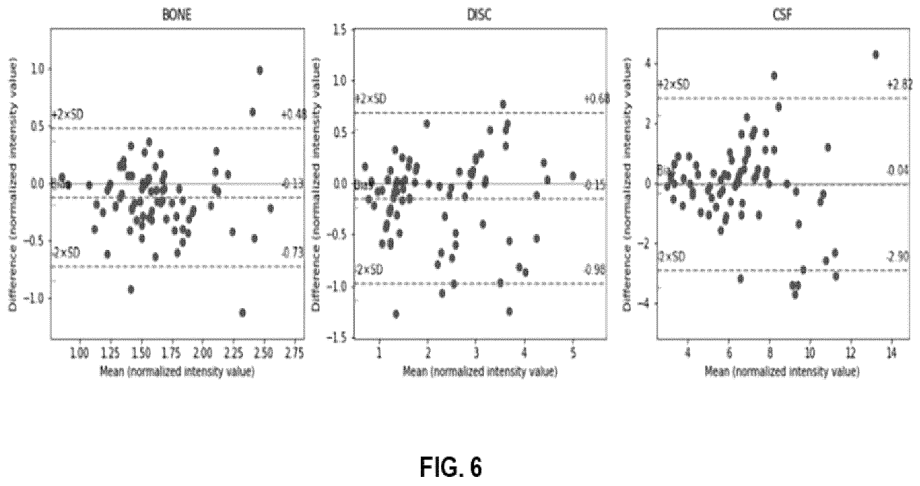

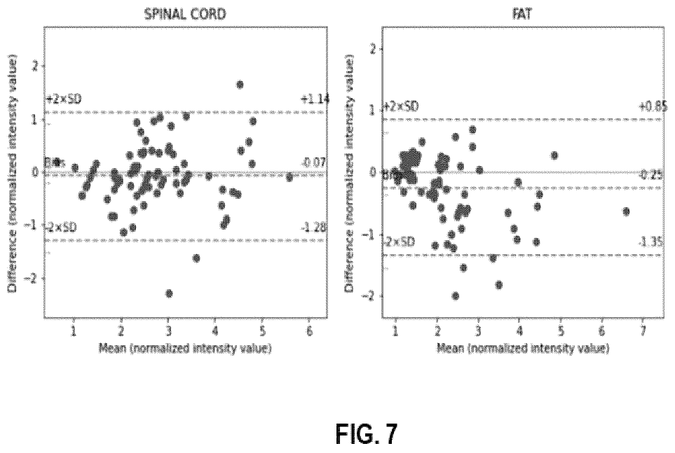

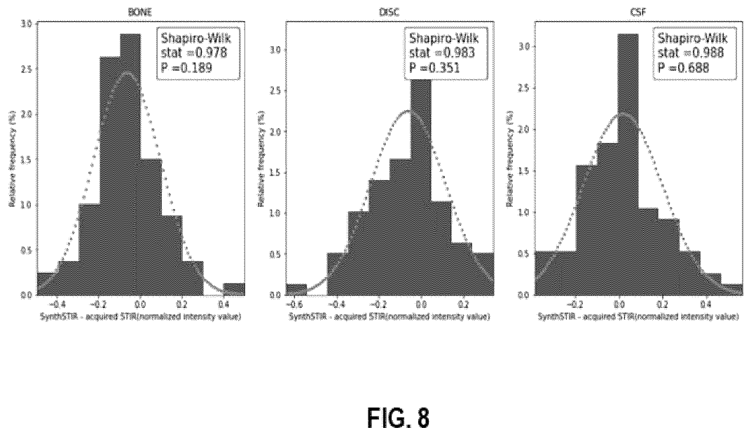

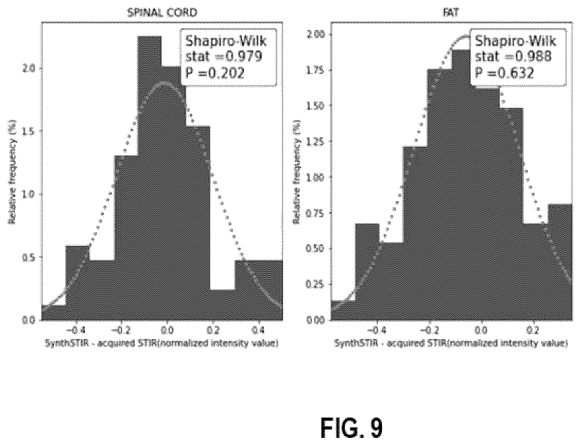

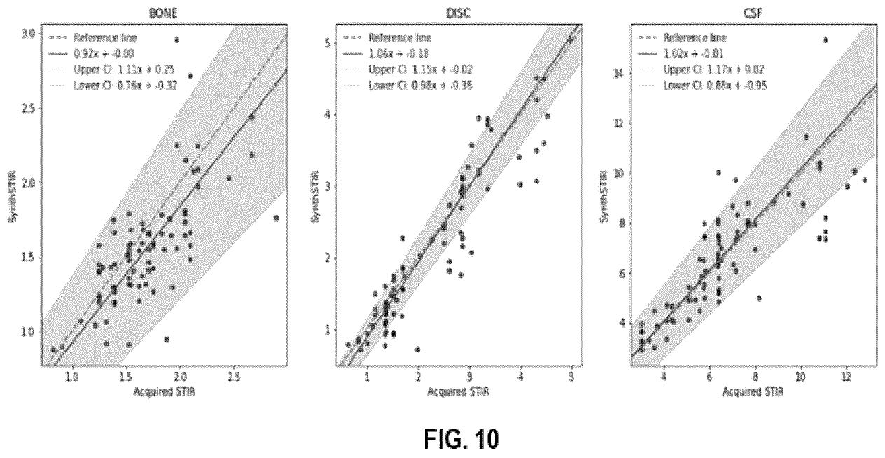

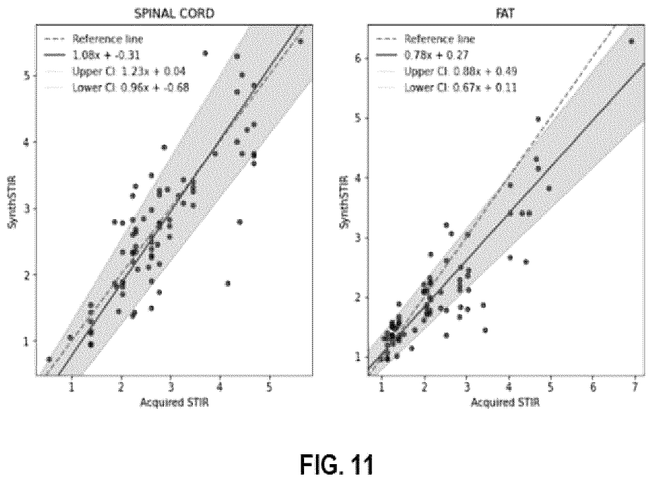

A study with 80 subjects undergoing clinical spine MRI exams (with Sagittal T1, Sagittal T2, Sagittal STIR) were applied to evaluate the pixel-wise correlation between the synthesis image and the acquired images. The Bland-Altman plots are shown in FIG. 6 and FIG. 7. The x-axis represents the mean of each tissue, and the y-axis represents the difference between the normalized intensity value from Synthesized STIR and its nearest neighbor from the acquired STIR. The x-axis represents the mean of the normalized intensity value from Synthesized STIR and its nearest neighbor from the acquired STIR, and the y-axis represents the difference between the normalized intensity value from Synthesized STIR and its nearest neighbor from the acquired STIR. For each tissue, the bias (the mean of the difference between the acquired STIR and Synthesized STIR) is close to zero. Also, the Shapiro-Wilk results showed that all the p-values were greater than 0.05, which implies the difference between the acquired STIR and the Synthesized STIR is normally distributed. FIG. 8 and FIG. 9 show the distribution plot of differences between the acquired STIR and Synthesized STIR. The dotted line represents Normal distribution. The statistical value of the Shapiro-Wilk test for each tissue and the corresponding p-value is put in the upper left. The slope of the disc, CSF, and spinal cord are all very close to 1, which indicates the high agreement between the synthesis image and the acquired image. For each tissue, the Passing-bablok regression was applied and its regression line and intercept are estimated. FIG. 10 and FIG. 11 show Passing-bablok regression plot for each tissue. The y-axis represents the normalized intensity value from the SynthSTIR, and the x-axis represents the normalized intensity value from its nearest neighbor from the acquired STIR.

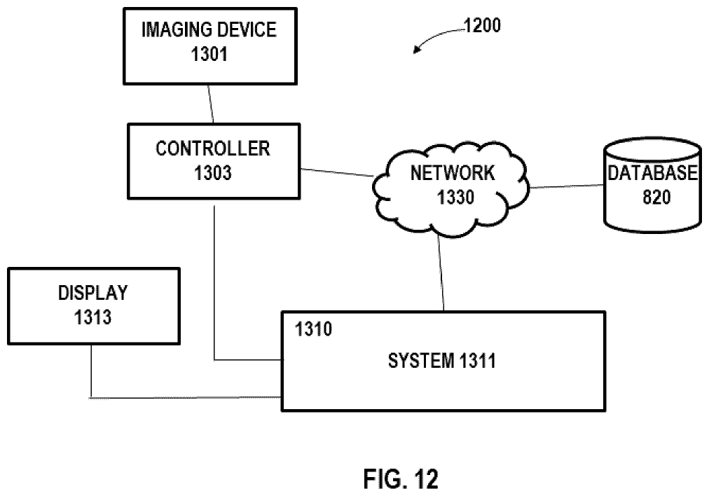

The systems and methods can be implemented on existing imaging systems without a need of a change of hardware infrastructure. In some embodiments, one or more functional modules for contrast synthesis may be provided as separate or self-contained packages. Alternatively, the one or more functional modules may be provided as an integral system. FIG. 12 schematically illustrates a system 1311 implemented on an imaging platform 1200 for performing one or more methods/algorithms described herein. The imaging platform 1200 may comprise a computer system 1310 and one or more databases 1320 operably coupled to a controller 1303 over the network 1330. The computer system 1310 may be used for implementing the methods and systems consistent with those described elsewhere herein to provide synthesizing a target or missing contrast(s), for example. The computer system 1310 may be used for implementing the system 1311. The system 1311 may include one or more functional modules such as the framework described in FIG. 1. The functional modules may be configured to execute programs to implement the reconstruction model for predicting the target contrast(s) as described elsewhere herein. Although the illustrated diagram shows the controller and computer system as separate components, the controller and computer system (at least some components of the system) can be integrated into a single component.

The system 1311 may comprise or be coupled to a user interface. The user interface may be configured to receive user input and output information to a user. The user interface may output a synthesized image of a target contrast generated by the system, for example, in real-time. The user interface may include a screen 1313 such as a touch screen and any other user interactive external device such as handheld controller, mouse, joystick, keyboard, trackball, touchpad, button, verbal commands, gesture-recognition, attitude sensor, thermal sensor, touch-capacitive sensors, foot switch, or any other device.

In some cases, the user interface may comprise a graphical user interface (GUI) allowing a user to view the synthesized image, and various other information generated based on the synthesized data. In some cases, the graphical user interface (GUI) or user interface may be provided on a display 1313. The display may or may not be a touchscreen. The display may be a light-emitting diode (LED) screen, organic light-emitting diode (OLED) screen, liquid crystal display (LCD) screen, plasma screen, or any other type of screen. The display may be configured to show a user interface (UI) or a graphical user interface (GUI) rendered through an application (e.g., via an application programming interface (API) executed on the local computer system or on the cloud). The display may be on a user device, or a display of the imaging system.

The imaging device 1301 may acquire image frames using any suitable imaging modalities live video or image frames may be streamed in using any medical imaging modality such as but not limited to MRI, CT, fMRI, SPECT, PET, ultrasound, etc. The acquired images may have missing data (e.g., due to corruption, degradation, low quality, limited scan time, etc.) such that the images may be processed by the system 1311 to generate the synthesized image data.

The controller 1303 may be in communication with the imaging device 1301, one or more displays 1313 and the system 1311. For example, the controller 1303 may be operated to provide the controller information to manage the operations of the imaging system, according to installed software programs. In some cases, the controller 1303 may be coupled to the system to adjust the one or more operation parameters of the imaging device based on a user input.

The controller 1303 may comprise or be coupled to an operator console which can include input devices (e.g., keyboard) and control panel and a display. For example, the controller may have input/output ports connected to a display, keyboard and other I/O devices. In some cases, the operator console may communicate through the network with a computer system that enables an operator to control the production and display of live video or images on a screen of display. In some cases, the image frames displayed on the display may be generated by the system 1311 (e.g., synthesized target contrast image(s)) or processed by the system 1311 and have improved quality.

The system 1311 may comprise multiple components as described above. In addition to the reconstruction model for synthesizing a target contrast image, the system may also comprise a training module configured to develop and train a deep learning framework using the training method and datasets as described above.

The computer system 1310 may be programmed or otherwise configured to implement the one or more components of the system 1311. The computer system 1310 may be programmed to implement methods consistent with the disclosure herein.

The imaging platform 1200 may comprise computer systems 1310 and database systems 1320, which may interact with the system 1311. The computer system may comprise a laptop computer, a desktop computer, a central server, distributed computing system, etc. The processor may be a hardware processor such as a central processing unit (CPU), a graphic processing unit (GPU), a general-purpose processing unit, which can be a single core or multi core processor, or a plurality of processors for parallel processing. The processor can be any suitable integrated circuits, such as computing platforms or microprocessors, logic devices and the like. Although the disclosure is described with reference to a processor, other types of integrated circuits and logic devices are also applicable. The processors or machines may not be limited by the data operation capabilities. The processors or machines may perform 512 bit, 256 bit, 128 bit, 64 bit, 32 bit, or 16 bit data operations.

The computer system 1310 can communicate with one or more remote computer systems through the network 1330. For instance, the computer system 1310 can communicate with a remote computer system of a user or a participating platform (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1310 or the system via the network 1330.

The imaging platform 1200 may comprise one or more databases 1320. The one or more databases 1320 may utilize any suitable database techniques. For instance, structured query language (SQL) or “NoSQL” database may be utilized for storing image data, collected raw data, attention scores, model output, synthesized image data, training datasets, trained model (e.g., hyper parameters), user specified parameters (e.g., window size), etc. Some of the databases may be implemented using various standard data-structures, such as an array, hash, (linked) list, struct, structured text file (e.g., XML), table, JSON, NOSQL and/or the like. Such data-structures may be stored in memory and/or in (structured) files. In another alternative, an object-oriented database may be used. Object databases can include a number of object collections that are grouped and/or linked together by common attributes; they may be related to other object collections by some common attributes. Object-oriented databases perform similarly to relational databases with the exception that objects are not just pieces of data but may have other types of functionality encapsulated within a given object. If the database of the present disclosure is implemented as a data-structure, the use of the database of the present disclosure may be integrated into another component such as the component of the present disclosure. Also, the database may be implemented as a mix of data structures, objects, and relational structures. Databases may be consolidated and/or distributed in variations through standard data processing techniques. Portions of databases, e.g., tables, may be exported and/or imported and thus decentralized and/or integrated.

The network 1330 may establish connections among the components in the imaging platform and a connection of the imaging system to external systems. The network 1330 may comprise any combination of local area and/or wide area networks using both wireless and/or wired communication systems. For example, the network 1330 may include the Internet, as well as mobile telephone networks. In one embodiment, the network 1330 uses standard communications technologies and/or protocols. Hence, the network 1330 may include links using technologies such as Ethernet, 802.11, worldwide interoperability for microwave access (WiMAX), 2G/3G/4G/5G mobile communications protocols, asynchronous transfer mode (ATM), InfiniBand, PCI Express Advanced Switching, etc. Other networking protocols used on the network 1330 can include multiprotocol label switching (MPLS), the transmission control protocol/Internet protocol (TCP/IP), the User Datagram Protocol (UDP), the hypertext transport protocol (HTTP), the simple mail transfer protocol (SMTP), the file transfer protocol (FTP), and the like. The data exchanged over the network can be represented using technologies and/or formats including image data in binary form (e.g., Portable Networks Graphics (PNG)), the hypertext markup language (HTML), the extensible markup language (XML), etc. In addition, all or some of links can be encrypted using conventional encryption technologies such as secure sockets layers (SSL), transport layer security (TLS), Internet Protocol security (IPsec), etc. In another embodiment, the entities on the network can use custom and/or dedicated data communications technologies instead of, or in addition to, the ones described above.

The methods or system herein may comprise any one or more of the abovementioned features, mechanisms and components or a combination thereof. Any one of the aforementioned components or mechanisms can be combined with any other components. The one or more of the abovementioned features, mechanisms and components can be implemented as a standalone component or implemented as an integral component.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein A and/or B encompasses one or more of A or B, and combinations thereof such as A and B. It will be understood that although the terms “first,” “second,” “third” etc. are used herein to describe various elements, components, regions and/or sections, these elements, components, regions and/or sections should not be limited by these terms. These terms are merely used to distinguish one element, component, region or section from another element, component, region or section. Thus, a first element, component, region or section discussed herein could be termed a second element, component, region or section without departing from the teachings of the present invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” or “includes” and/or “including,” when used in this specification, specify the presence of stated features, regions, integers, steps, operations, elements and/or components, but do not preclude the presence or addition of one or more other features, regions, integers, steps, operations, elements, components and/or groups thereof.

Reference throughout this specification to “some embodiments,” or “an embodiment,” means that a particular feature, structure, or characteristic described in connection with the embodiment is included in at least one embodiment. Thus, the appearances of the phrase “in some embodiment,” or “in an embodiment,” in various places throughout this specification are not necessarily all referring to the same embodiment. Furthermore, the particular features, structures, or characteristics may be combined in any suitable manner in one or more embodiments.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A computer-implemented method for synthesizing a contrast-weighted image comprising:
(a) receiving a multi-contrast image of a subject, wherein the multi-contrast image comprises one or more acquired images of one or more different contrasts, and wherein the one or more different contrasts correspond to one or more different pulse sequences for acquiring the multi-contrast image;
(b) generating an input data to be processed by a deep learning model, wherein the deep learning model is trained using a training data pair, wherein the training data pair includes an input image or a ground truth image, and wherein the input image or the ground truth image is adjusted based on a segmentation of a region of interest (ROI); and
(c) generating, by the deep learning model, a synthesized image based on the input data, wherein the synthesized image has a target contrast that is different from the one or more different contrasts of the one or more acquired images.

2. The computer-implemented method of claim 1, wherein generating the input data comprises registering the one or more acquired images.

3. The computer-implemented method of claim 2, wherein registering the one or more acquired images comprises adjusting at least one of the one or more acquired images based on a segmentation of a ROI.

4. The computer-implemented method of claim 3, wherein adjusting the at least one of the one or more acquired images comprises replacing the ROI in the at least one of the one or more acquired images with the segmentation of the ROI.

5. The computer-implemented method of claim 4, wherein the ROI contains motion of an anatomical region within the ROI.

6. The computer-implemented method of claim 1, wherein the deep learning model is trained by a framework comprising a segmentation network for generating a segmentation map, and a classification network for generating a pathology map.

7. The computer-implemented method of claim 6, wherein the segmentation map, and the pathology map are used to train the deep learning model.

8. The computer-implemented method of claim 6, wherein the ground truth image is adjusted by generating different brightness levels in a tissue area based on the segmentation of the ROI generated by the segmentation network.

9. The computer-implemented method of claim 6, wherein the input image of the training data pair is adjusted by replacing the ROI in the input image based on the segmentation of the ROI.

10. The computer-implemented method of claim 6, wherein the segmentation map is embedded into a loss function to train the deep learning model.

11. The computer-implemented method of claim 6, wherein the pathology map is embedded into a loss function to train the deep learning model.

12. The computer-implemented method of claim 1, wherein the multi-contrast image is acquired using a magnetic resonance (MR) device.

13. A non-transitory computer-readable storage medium including instructions that, when executed by one or more processors, cause the one or more processors to perform operations comprising:
(a) receiving a multi-contrast image of a subject, wherein the multi-contrast image comprises one or more acquired images of one or more different contrasts, and wherein the one or more different contrasts correspond to one or more different pulse sequences for acquiring the multi-contrast image;
(b) generating an input data to be processed by a deep learning model, wherein the deep learning model is trained using a training data pair, wherein the training data pair includes an input image or a ground truth image, and wherein the input image or the ground truth image is adjusted based on a segmentation of a region of interest (ROI); and
(c) generating, by the deep learning model, a synthesized image based on the input data, wherein the synthesized image has a target contrast that is different from the one or more different contrasts of the one or more acquired images.

14. The non-transitory computer-readable storage medium of claim 13, wherein generating the input data comprises registering the one or more acquired images.

15. The non-transitory computer-readable storage medium of claim 14, wherein registering the one or more acquired images comprises adjusting at least one of the one or more acquired images based on a segmentation of a ROI.

16. The non-transitory computer-readable storage medium of claim 15, wherein adjusting the at least one of the one or more acquired images comprises replacing the ROI in the at least one of the one or more acquired images with the segmentation of the ROI.

17. The non-transitory computer-readable storage medium of claim 16, wherein the ROI contains motion of an anatomical region within the ROI.

18. The non-transitory computer-readable storage medium of claim 13, wherein the deep learning model is trained by a framework comprising a segmentation network for generating a segmentation map, and a classification network for generating a pathology map.

19. The non-transitory computer-readable storage medium of claim 18, wherein the segmentation map, and the pathology map are used to train the deep learning model.

20. The non-transitory computer-readable storage medium of claim 18, wherein the ground truth image is adjusted by generating different brightness levels in a tissue area based on the segmentation of the ROI generated by the segmentation network.
